(19) United States
(12) Patent Application Publication
(10) Pub. No.: US 2009/0203116 A1
(43) Pub. Date: Aug. 13, 2009

(54) System to improve algae production in a photo-bioreactor

(76) Inventor: Keith E. Bazaire, Bradenton Beach, FL (US)

Correspondence Address: Keith Bazaire, 1900 Gulf Dr. N. # 7, Bradenton Beach, FL, 34217, US

(21) Appl. No.: 12/069,892

(22) Filed: Feb. 13, 2008

(51) Int. Cl. C12N 1/12; C12M 1/00

(52) U.S. Cl. 435/257.1; 435/292.1

(57) ABSTRACT

This invention provides a method of delivering light internally to a Photo-Bioreactor growing algae for production of Biodiesel fuel. The invention also provides an improved method of delivering liquid nutrients [I.e. sewage] and carbon dioxide to the growing algae in a Photo-Bioreactor. These results are achieved by a single system which delivers fiber optic light as well as liquid nutrients and carbon dioxide via jets. The net result attained by using this invention is a significant increase in the production of valuable algae with a very small increase in energy consumption. The increase in energy usage is the electrical energy required to power a 150 watt metal-halide bulb.

### CROSS REFERENCE TO RELATED APPLICATIONS

“Not Applicable”

### STATEMENT REGARDING FEDERALLY SPONSORED RESEARCH OR DEVELOPMENT

“Not Applicable”

### REFERENCE TO SEQUENCE LISTING, A TABLE, OR A COMPUTER PROGRAM LISTING COMPACT DISC APPENDIX

“Not Applicable”

### BACKGROUND OF THE INVENTION

This invention pertains to the production of Biodiesel fuel. It specifically pertains to the production of Algae as a source of oil which can be converted to Biodiesel through transesterification. Algae can be grown in a system called a Photo-Bioreactor which are currently being manufactured. The current technology has two major problems which are solved by this invention.

The current Photo-Bioreactors rely solely on external light sources, sunlight or artificial lights, with the light penetrating the reactor contents through the transparent reactor tube. As the Algae grows inside the reactor the external light is blocked by the algae and cannot penetrate to the center of the reactor. This blocking effect retards the rate of growth of the algae throughout the reactor. Since algae depends on light to grow, this lack of penetration of light to the center limit's the productivity of the current Photo-Bioreactors.

This invention solves this problem by providing a way to deliver light to the center of the reactor using fiber optic cables as an internal source of light. By wrapping the cable around a plastic pipe at the center of the reactor the fiber optic light can be emitted 360 degrees along the entire length of the reactor tube. The amount of light provided in this manner is determined by the size and number of fiber optic cables that are wrapped around the center pipe.

A second problem with the current technology is the difficulty of maintaining steady flow of the algae through the reactor as well as inability to maintain good contact of the required nutrients with the growing algae. This invention, by delivering the liquid nutrients to the growing algae through jets, solves both of the problems. The action of the jets will cause stirring of the contents of the reactor resulting in better contact of the nutrients with the growing algae. Also, by directing the discharge of the jets toward the direction of flow of the process stream the force of the discharge will propel the algae downstream without the use of mechanical force. The discharge of the jets will also impinge on the inner surface of the reactor tube and keep algae from accumulating on the surface where it can further retard the penetration of external light.

### BRIEF SUMMARY OF THE INVENTION

The object of this invention is to enhance the productivity of current Photo-Bioreactors used to grow algae for Biodiesel production. This invention consists of a length of plastic pipe wrapped with fiber optic side glow cables and fitted with jets for injection of carbon dioxide and liquid nutrients into the algae in water contents of a Photo-Bioreactor. This device solves the problems of limited light penetration from external sources and limited contact of nutrients with the algae growing inside the Photo-Bioreactor.

### BRIEF DESCRIPTION OF THE SEVERAL VIEWS OF THE DRAWING

The drawings show how the invention is assembled and how it is installed on the existing Photo-Bioreactor systems. FIG. 1 shows the invention as it would be mounted on an existing continuous flow Photo-Bioreactor [1]. An extension [2] is affixed to the curved end of a reactor tube. This extension has a flange [3] with an O-Ring seal [4]. The flange is drilled to allow for bolting the fiber optic wrapped nutrient feed pipe assembly [5] to it. When assembled, as shown in FIG. 1 the fiber optic pipe assembly [5] will be centered in the lateral part of the Photo-Bioreactor tube. Bolts are used to complete the assembly and the O-Ring prevents leakage of liquid from the Photo-Bioreactor. The fiber optic light source [6] is also depicted in FIG. 1. This light source uses a metal-halide bulb and is capable of providing light to several fiber optic cables.

FIG. 2 shows a top view of a portion [several loops] of a continuous flow Photo-Bioreactor with the orientation of the fiber optic wrapped nutrient feed pipes depicted. This view also shows how the jets [7] are located along the horizontal axis of the pipes. The arrows on the depicted jets indicate the direction of discharge from the jets to enhance movement of the algae down the tube and cleaning of the surface of the reactor tubes. The diagonal lines on the nutrient feed pipes depict the fiber optic cables [8] wrapped around the pipes.

The feed sources for liquid nutrient [9] and carbon dioxide [10] are depicted in FIG. 2. Also, fiber optic light sources [6] are depicted with multiple fiber optic cables leading to the assembly.

FIG. 3 shows a top view of the fiber optic wrapped nutrient feed pipe assembly [5]. The inlets for liquid nutrient [11] and carbon dioxide [12] are shown. The bolts [13] which attach the assembly to the flange on the extension of the Photo-Bioreactor are shown in the end plate [14]. Also shown on the end plate is a connector [15] for the fiber optic cable [8] which passes through the end plate and is wrapped around the nutrient feed pipe.

The nutrient feed pipe is mounted on the end plate [14] in a sleeve [16] which is glued to the inside face of the end plate [14]. The pipe [5] is glued into the sleeve [16]. A fiber optic light source [6] is depicted with a fiber optic cable [8] in this view.

FIG. 4 shows a top view of the extension [2] of the Photo-Bioreactor [I] with the mounting flange [3] and O-Ring [4].

FIG. 5 is an end view of the nutrient feed pipe [5] inside the Photo-Bioreactor [1]. This view shows the orientation of the nutrient feed jets [7] around the circumference of the nutrient feed pipe.

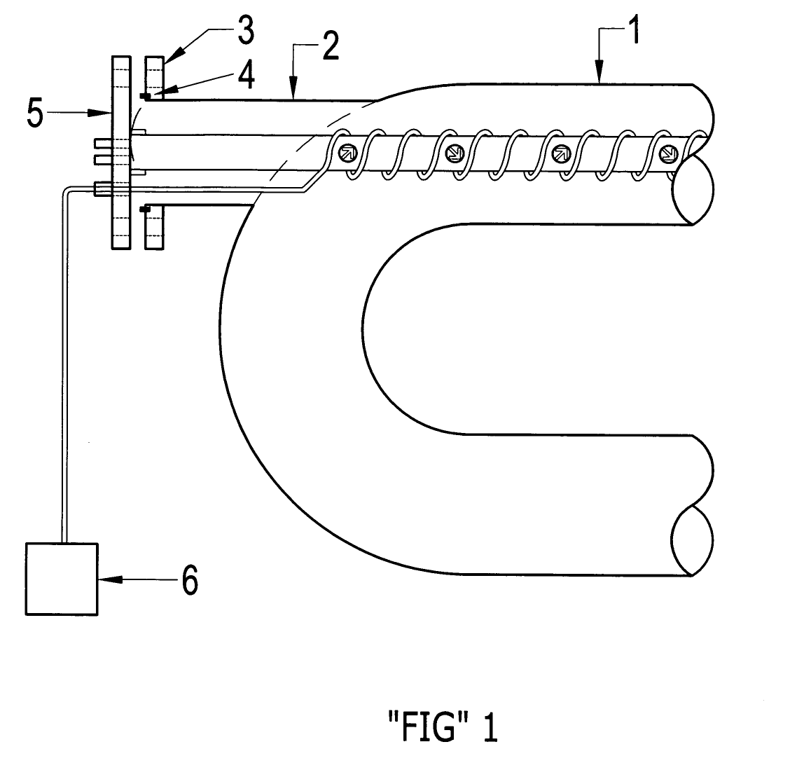

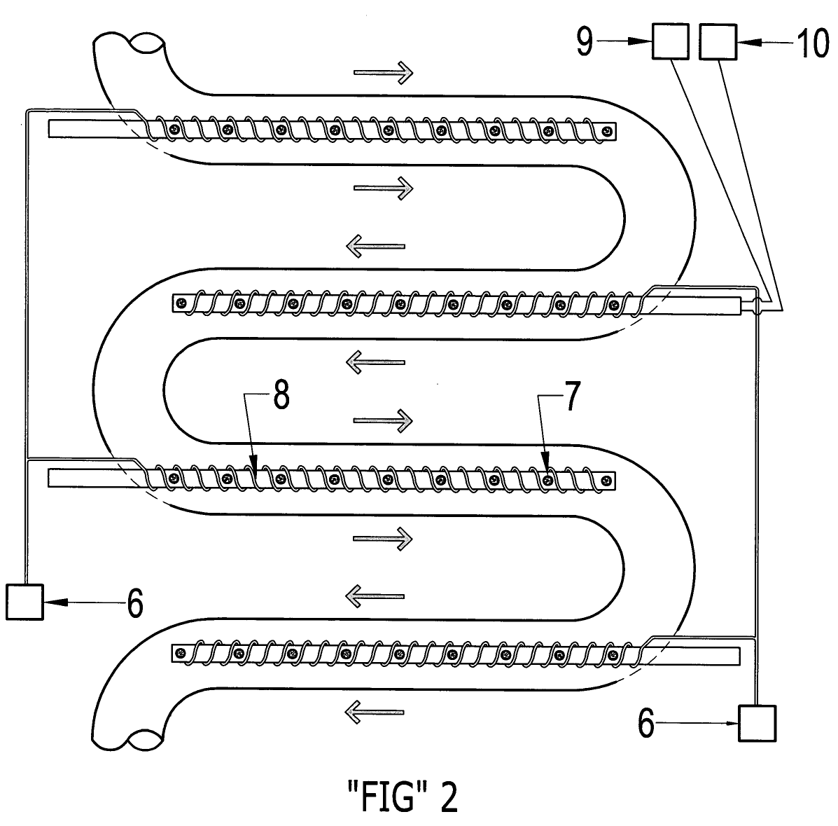

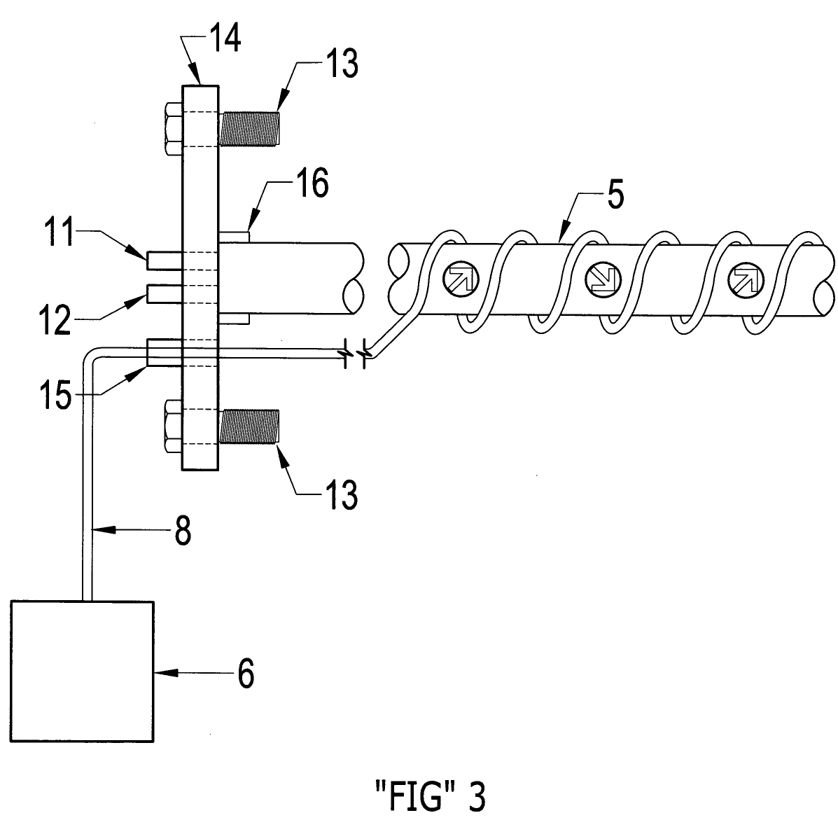

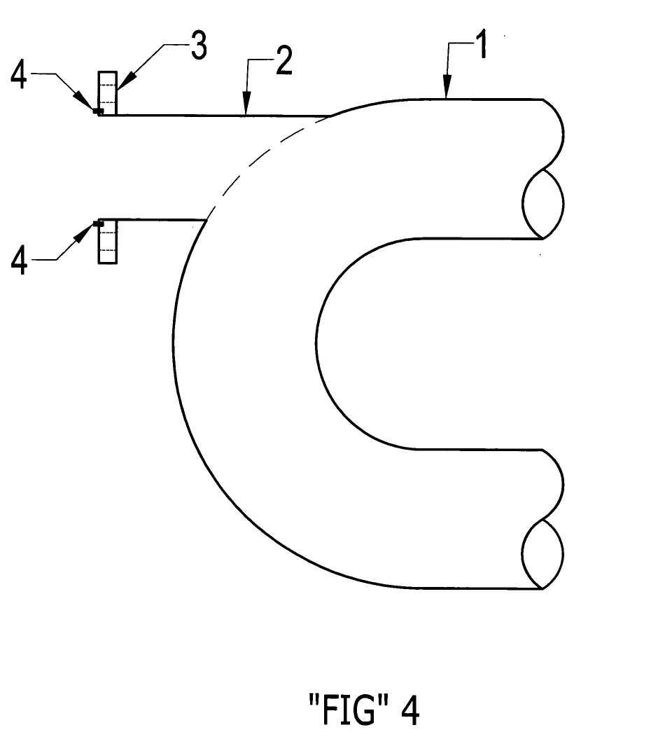

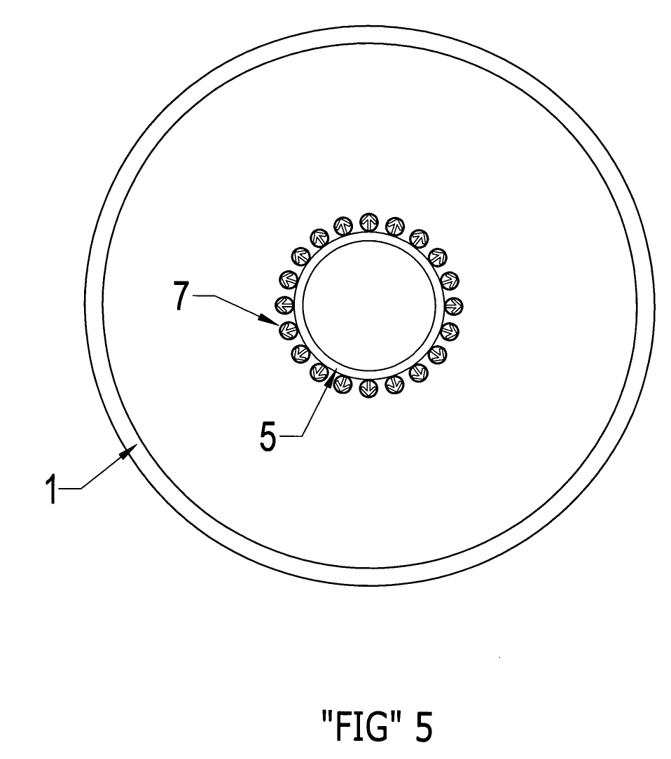

### DETAILED DESCRIPTION OF THE INVENTION

This invention uses readily available components which are assembled to create a method of providing light internally to a Photo-Bioreactor in which algae is grown for use as a source of oil to be converted to Biodiesel fuel. The assembly also provides a better method of injecting liquid nutrients and carbon dioxide into the growing algae.

This invention uses a length of plastic pipe [common PVC plumbing pipe] which is attached to a plastic end plate which is drilled for bolts so it can be bolted to a plastic flange on an extension of a Photo-Bioreactor tube. One end of the plastic pipe is attached to the inside face of the plastic end plate by gluing it into a plastic sleeve which is glued to the endplate. Plastic [PVC] fittings are also attached to the outside face of the endplate to provide inlets for liquid nutrients and carbon dioxide. A fiber optic coupler is also attached to the outside face of the endplate to allow for fiber optic cable to be fed through the endplate and then wrapped around the plastic pipe.

To provide for liquid nutrient feed and carbon dioxide injection into the Photo-Bioreactor jets are mounted on the outer surface of the plastic pipe. These jets are available from Spa/Hot Tub equipment suppliers. They also are made of plastic materials.

The fiber optic cables, connectors, and light sources are available from suppliers of fiber optic devices. The fiber optic cables are made of clear transparent plastic. The type used in this invention is the “stranded side glow version” [I.e. SG100]. The light source uses a metal-halide bulb in a unit designed to supply light to several fiber optic cables [I.e. FOP150-Encore].

This invention can be used to improve the productivity of any type of Photo-Bioreactor [I.e. Continuous flow, batch tank, open vat, open pond, etc.]. It is not limited to use with continuous flow reactors. The improvement to current reactor systems is attained by delivering internal light via fiber optics and by the agitation provided by the jets supplying the liquid nutrients and carbon dioxide.

In addition to supplementing the external light sources during daylight hours, this invention also provides a source of light during hours of darkness. This further increases the productivity of the Photo-Bioreactor by providing light for algae growth 24 hrs/day.

1. The rate of growth and daily production of algae for use in the production of Biodiesel fuel will be significantly increased by modifying existing Photo-Bioreactors with this system.

2. This invention provides a unique method of delivering light, which is necessary for the growth of algae, internally in a Photo-Bioreactor.

3. The system of jets injecting liquid nutrients and carbon dioxide into the Photo-Bioreactor will increase the rate of growth of algae through more efficient consumption of nutrients by the growing algae.

4. This invention can improve the growth of algae in any Photo-Bioreactor [I.e. continuous flow, tank open vat, pond, etc.].

5. This system can be used to supplement the use of natural light or completely replace natural light in a Photo-Bioreactor in which algae is grown.
